(19) United States
(12) Patent Application Publication
(10) Pub. No.: US 2011/0033904 A1
(43) Pub. Date: Feb. 10, 2011

(54) METHOD FOR PREPARING ORTHODIHYDROXYISOFLAVONES USING A BIOTRANSFORMATION SYSTEM

(76) Inventors: Jun Seong Park, Gyeonggi-do (KR); Hye Yoon Park, Gyeonggi-do (KR); Ho Sik Rho, Gyeonggi-do (KR); Duck Hee Kim, Seoul (KR); Ih Seop Chang, Gyeonggi-do (KR); Byung Gee Kim, Seoul (KR); Chang Hyun Roh, Daejeon (KR); Su Hyun Seo, Seoul (KR); Kwon Young Choi, Seoul (KR); June Hyung Kim, Seoul (KR); Min-Ho Cha, Seoul (KR); Prasad Pandey Bishnu, Rupandehi (NP)

Correspondence Address: NIXON & VANDERHYE, PC, 901 NORTH GLEBE ROAD, 11TH FLOOR, ARLINGTON, VA, 22203, US

(21) Appl. No.: 12/937,876

(22) PCT Filed: Apr. 25, 2008
(86) PCT No.: PCT/KR2008/002375

§ 371 Date: Oct. 14, 2010

(51) Int. Cl. C12P 17/06

(52) U.S. Cl. 435/125

(57) ABSTRACT

Disclosed herein is a method for preparing ortho-dihydroxyisoflavones using a biotransformation system. More specifically, disclosed is a method for preparing ortho-dihydroxyisoflavones, which comprises biotransforming daidzein or genistein by actinomycete microorganisms, particularly Streptomyces avermitilis, Nocardia farcinica or Streptomyces lincolnesis, in order to efficiently prepare ortho-dihydroxyisoflavones having an excellent antioxidant function and a whitening effect.

### TECHNICAL FIELD

The present invention relates to a method for preparing ortho-dihydroxyisoflavones using a biotransformation system, and more particularly to a method for preparing ortho-dihydroxyisoflavones, which comprises biotransforming daidzein or genistein by actinomycete microorganisms, particularly Streptomyces avermitilis, Nocardia farcinica or Streptomyces lincolnesis, in order to efficiently prepare ortho-dihydroxyisoflavones having an excellent antioxidant function and a whitening effect.

### BACKGROUND ART

Isoflavones which are vegetable compounds contained mainly in beans are present as glucosides containing isoflavones as aglycons and are transformed into aglycons by microbial metabolism during fermentation processes. Isoflavones are known to have anticancer, antioxidant, antiatherogenic, blood glucose-lowering and osteoporosis-preventing effects, and thus studies thereon are being actively conducted. Among isoflavones, particularly 7,8,4′-trihydroxyisoflavone, 7,6,4′-trihydroxyisoflavone, 7,3′,4′-trihydroxyisoflavone and 7,5,3′,4′-tetrahydroxyisoflavone, which are ortho-dihydroxyisoflavones (ODIs), are highly valuable, because they have high antioxidant effects compared to those of other isoflavones and are difficult to synthesize chemically.

Daidzein and genistein are diphenolic phytoestrogen compounds found in numerous plants and soybeans. Such compounds were reported to have antioxidant, antimicrobial and metal chelating effects (Middleton et al., 1992; Dixon et al., 2002). Furthermore, they are being used as medical or chemical therapeutic agents for human health (Foti et al., 2005). Recently, interest in position-specific hydroxylated compounds of daidzein or genistein has increased. Ortho-specific hydroxylated isoflavones were reported to have biological effects higher than those of daidzein or genistein (Rufer and Kulling, 2006) and are known to anti-inflammatory and anti-allergenic activities (Rufer and Kulling, 2006). Also, they are tyrosine kinase inhibitors, have anticarcinogenic properties (Akiyama et al., 1987) and are used as potent tyrosinase inhibitors and lipoxygenase inhibitors (Chang et al., 2005; Voss et al., 1992). In addition, the use thereof as compounds for reducing the cause of cancer-related diseases was reported (Klus and Barz, 1995; Coward et al., 1993). Such hydroxylated compounds are difficult to synthesize by organic chemical methods, and thus can be produced only by either extraction from natural materials or biosynthesis methods.

As described above, hydroxylated isoflavones can be isolated and purified only by either extraction from natural materials or biosynthesis using microorganisms. In the case of extraction from natural materials, daidzein and genistein are easily obtained as the main compounds of isoflavones, but the hydroxylated forms of daidzein and genistein are not so. In other words, ortho-dihydroxyisoflavones, which are contained in soybeans or plants are obtained in low yield, and the biosynthesis thereof using microorganisms have also not been much studied.

In the prior art, hydroxylated compounds extracted from natural materials in low yield, and hydroxylated compounds obtained using microorganisms having low reactivity were analyzed, but there was no report of the productivity of ortho-dihydroxyisoflavones by microorganisms. Also, the reactivity of ortho-dihydroxyisoflavones with animal liver cells was reported, but there was a problem of low productivity due to low reactivity (Kulling et al, 2001).

### DISCLOSURE

### Technical Problem

Accordingly, the present inventors have found that ortho-dihydroxyisoflavones having an antioxidant function and a whitening effect can be efficiently prepared by biotransforming daidzein and genistein by actinomycete microorganisms, particularly Streptomyces avermitilis, Nocardia farcinica or Streptomyces lincolnesis, thereby completing the present invention.

It is, therefore, an object of the present invention to provide a method of preparing ortho-dihydroxyisoflavones by biotransforming daidzein and genistein.

### Technical Solution

To achieve the above object, the present invention provides a method of preparing ortho-dihydroxyisoflavones by biotransforming daidzein and genistein by actinomycete microorganisms, particularly Streptomyces sp. or Nocardia sp. microorganisms.

### ADVANTAGEOUS EFFECTS

According to the present invention, there can be provided a preparation method of specifically producing and accumulating ODIs, which are antioxidant substances or whitening substances, using actinomycete microorganisms, and furthermore, biosynthesizing modified compounds. The present invention will become a high value-added invention in scientific research and industrial application.

### DESCRIPTION OF DRAWINGS

FIG. 1 shows the results of HPLC analysis of each test substance. (1): 7,3′,4′-trihydroxyisoflavone as a metabolite; peak (2): daidzein as a substrate; peak (3): 7,5,3′,4′-tetrahydroxyisoflavone as a metabolite; and peak (4): genistein as a substrate. The HPLC analysis was performed in the following conditions: UV: 254 nm; flow rate: 1 ml/min; and solvent used: ACN:DW=3:7 (containing 1% TFA).

FIG. 2 shows the results of NMR analysis of 7,3′,4′-trihydroxyisoflavone.

FIG. 3 shows the results of NMR analysis of 7,5,3′,4′-tetrahydroxyisoflavone.

FIG. 4 shows reactors for biotransforming daidzein using Streptomyces avermitilis. (A): a prior art reactor; and (B): a batch reactor used in the present invention to make oxygen supply smooth.

FIG. 5 shows the results of the GC-MS analysis of substrate daidzein and reaction products, carried out using BSTFA derivatization. 1): daidzein, room temperature, 18.5 min, MS 398; 2) 7,3′,4′-trihydroxyisoflavone, room temperature, 24.4 min, MS 486; 3) 7,8,4′-trihydroxyisoflavone, room temperature, 25.1 min, MS 486; and 4) 7,6,4′-trihydroxyisoflavone, room temperature, 26.8 min, MS 486.

FIG. 6 shows the structures of various modified isoflavones produced by biotransforming daidzein by Streptomyces avermitilis.

FIG. 7 shows the results of the reaction of Streptomyces avermitilis with daidzein and the results of GC analysis (reaction conditions: use of coils, 220 rpm, reaction time: 1 month, and use of R2YE medium).

FIG. 8 is a schematic diagram showing a process of derivatization by BSTFA for analysis.

FIGS. 9 and 10 show the results of MS spectrum analysis of test substances.

### BEST MODE

Hereinafter, the present invention will be described in further detail.

The present invention relates to a method of preparing desired compounds, that is, ortho-specific hydroxylated isoflavones, using actinomycete microorganisms. In the present invention, microbial strains capable of preparing ortho-dihydroxyisoflavones using daidzein and genistein as substrates were first screened.

Microbial strains which screened in the present invention include Streptomyces avermitilis, Nocardia farcinica and Streptomyces lincolnesis. Among these strains, Streptomyces avermitilis showed the highest ortho-dihydroxyisoflavone productivity and also showed the position-specific hydroxylation at the 3′ position of genistein.

Although the terms used in the present invention are conventionally used in the art and the meaning thereof can be understood by any person skilled in the art, the terms are briefly defined as follows:

1) ODI: ortho-dihydroxyisoflavone.

2) ISP2 medium (per 1 liter): 5 g malt extract, 2 g yeast extract and 2 g glucose.

3) YEME medium (per 1 liter): 3 g malt extract, 3 g yeast extract, 5 g peptone, 300 g sucrose, 2 ml MgCl₂.6H₂O (2.5M) and 25 ml glycine (20%).

4) R2YE medium: 103 g sucrose, 10 g glucose, 0.25 g K₂SO₄, 5 g yeast extract, 0.1 g Difco casamino acid, 100 ml TES buffer (5.73%, pH 7.2), 10 ml KH₂PO₄ (0.5%), 80 ml CaCl₂.2H₂O (3.68%), 15 ml L-proline (20%), 2 ml trace element solution and 5 ml NaOH (1N).

5) HPLC: High-Performance Liquid Chromatography.

6) GC-MS: Gas Chromatography-Mass Spectrometry.

7) NMR: Nuclear Magnetic Resonance spectroscopy.

8) BSTFA: a derivative for GC analysis. N,O-bis(trimethylsilyl)trifluoroacetamide).

9) rpm: revolutions per minute or the revolutions per minute of a disc.

The method according to the present invention comprises a step of biotransforming daidzein and genistein by the screened strains. Namely, the present invention comprises a biotransformation process of the following reaction scheme 1 of preparing 3′-specific hydroxylated compounds from daidzein and genistein using the screened strains:

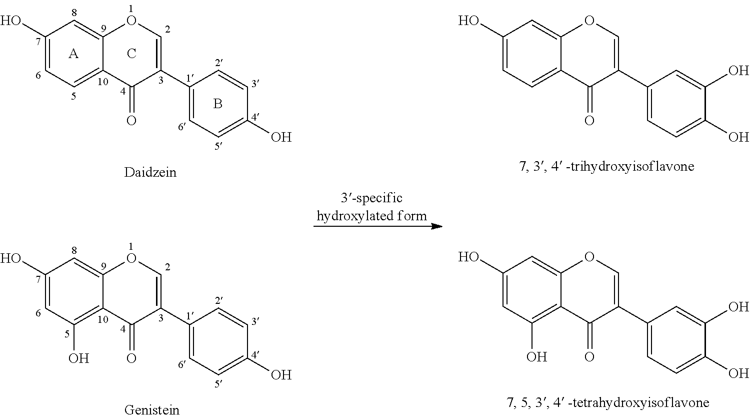

The following reaction scheme 2 shows a process of producing ortho-specific hydroxylated compounds from daidzein and genistein, which are the typical constituent compounds of isoflavones:

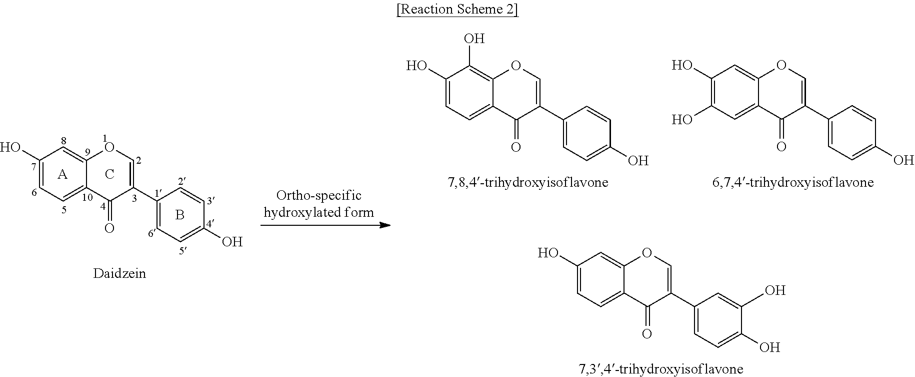

Ortho-dihydroxyisoflavones produced according to the present invention include 7,8,4′-trihydroxyisoflavone, 7,6,4′-trihydroxyisoflavone, 7,3′,4′-trihydroxyisoflavone and 7,5,3′,4′-tetrahydroxyisoflavone.

According to the present invention, four desired position-specific forms of a desired compound, 7,8,4′-trihydroxyisoflavone, 7,6,4′-trihydroxyisoflavone, 7,3′,4′-trihydroxyisoflavone and 7,5,3′,4′-tetrahydroxyisoflavone (Orobol), could be produced using microbial strains having high substrate-specific reactivity (in the case like Orobol, biosynthesis using microorganisms is the first in the world).

In the present invention, ortho-dihydroxyisoflavones could be produced using a modified reactor and reaction time.

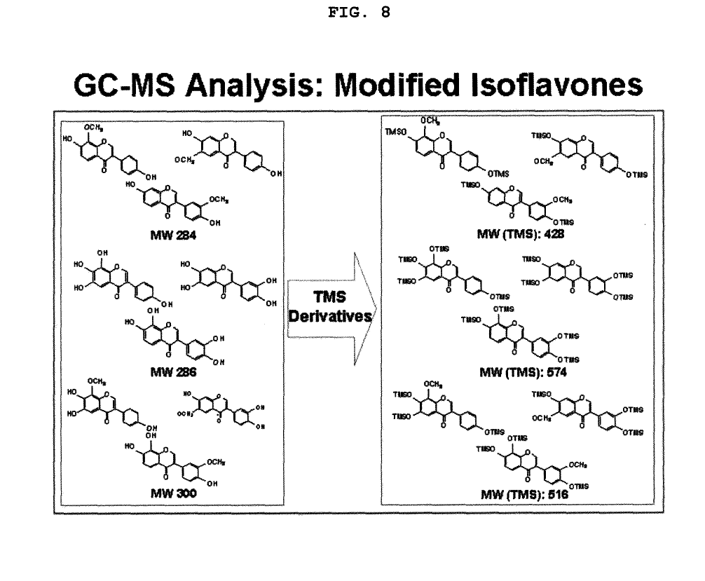

Reaction conditions according to the present invention are as follows: For smooth oxygen supply to a microbial strain during a reaction, coils or glass beads are used in a batch reactor. A microbial strain primarily cultured in a test tube is subcultured in a 1-liter conical flask for 48 hours, and then the reactivity thereof is examined using a fresh medium. Herein, the medium is preferably ISP2, YEME or R2YE. Among them, R2YE is preferably in view of the growth reactivity of the strain. The reaction time is examined at an interval of 12 hours. When the reaction was carried out for 24 hours, the production of 7,3′,4′-trihydroxyisoflavone and 7,5,3′,4′-tetrahydroxyisoflavone (Orobol) was increased, and after 36 hours of the reaction, the production of 7,8,4′-trihydroxyisoflavone and 7,6,4′-trihydroxyisoflavone was increased, and after 48 hours of the reaction, modified isoflavones could be obtained (FIG. 8). The concentration (per reaction volume) of substrate used is 10 mM, and the culture of the strain and the reaction are carried out 28° C.

The ortho-dihydroxyisoflavones according to the present invention are polar compounds having antioxidant or whitening effects.

According to the present invention, the amount of ortho-dihydroxyisoflavones and modified isoflavones, which are rarely present in vegetable materials, can be greatly increased through biosynthesis using, as a raw material, daidzein or genistein which are the constituent components of most isoflavones.

Daidzein and genistein, used as substrates in the present invention, were purchased from Bioland Co., Ltd. (Korea).

In the present invention, Streptomyces avermitilis which is an actinomycete microorganism is cultured and examined for reactivity with daidzein. Based on the examination results, ortho-dihydroxyisoflavone compounds are collected and purified from the cultured material using the specific gravity difference of ethyl acetate (EA). In other words, by examining the reactivity of daidzein with actinomycete Streptomyces avermitilis, the production of orthodihydroxyisoflavones can be accumulated and the antioxidant ortho-dihydroxyisoflavones can be prepared from the cultured material.

### MODE FOR INVENTION

Hereinafter, the present invention will be described in further detail with reference to examples. It is to be understood, however, that these examples are for illustrative purposes and are not to be construed to limit the scope of the present invention.

### Example 1

### Examination of Reactivity of Daidzein and Genistein from Gram-Positive Streptomyces Avermitilis

Microorganisms performing a hydroxylation reaction of attaching an —OH group specifically at the 6, 8 or 3′ position of daidzein were screened and the enzymes thereof were examined.

First, substrate specificities for daidzein and genistein were examined with yeasts, fungi and bacteria, which had different compositions. Among the examined microbial strains, Streptomyces Avermitilis, Nocardia farcinica and Streptomyces lincolnesis showed reactivity with daidzein. Among them, Streptomyces Avermitilis showed the highest substrate specificity for daidzein and genistein.

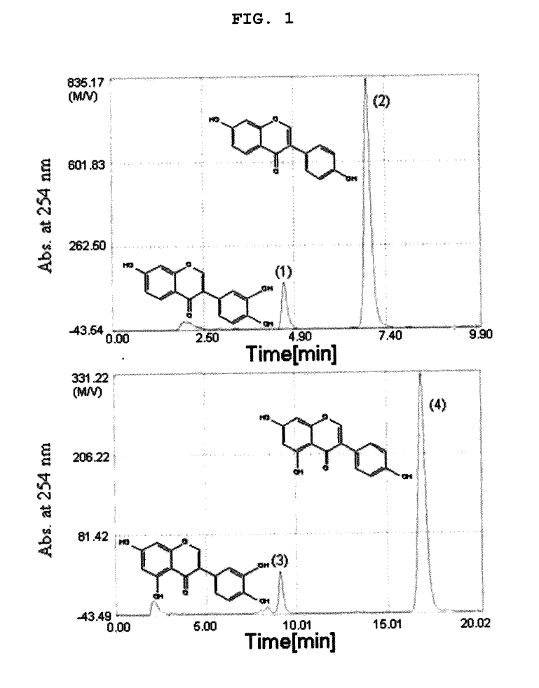

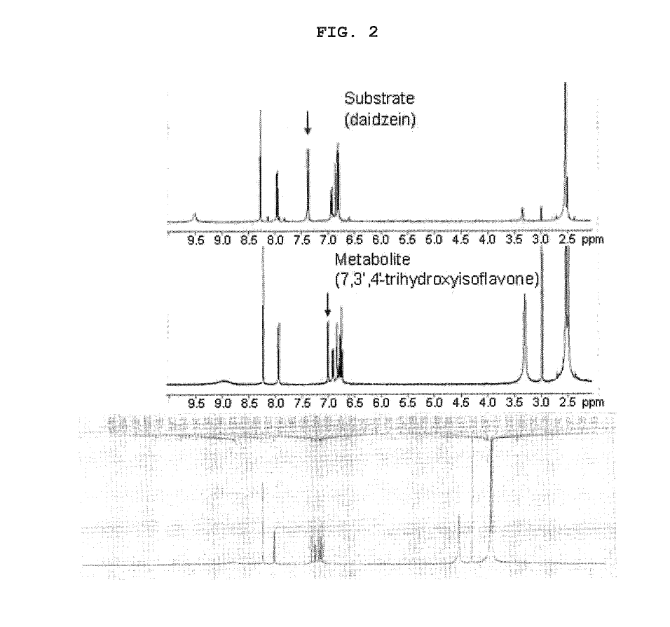

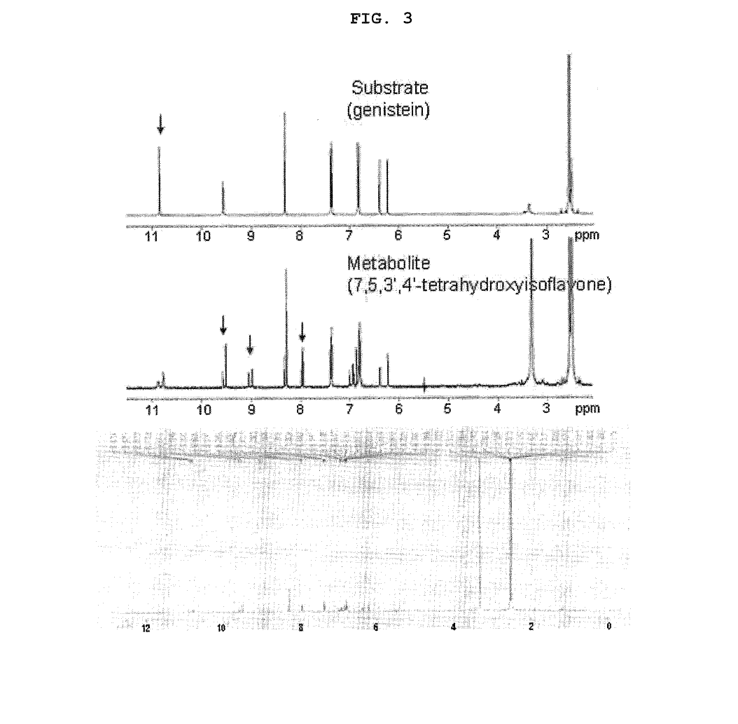

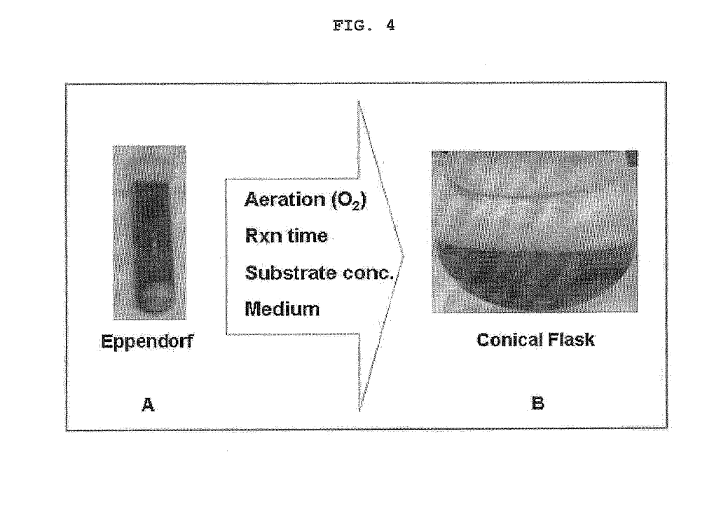

As the reaction medium, an ISP2 medium was used, and a batch reactor shown in FIG. 4 was used instead of Eppendorf in the reaction. After 24 hours of the reaction, the reaction products were extracted with ethyl acetate, evaporated using a vacuum centrifuge, and then analyzed. The structures of the reaction products were analyzed using HPLC and NMR analysis systems, and the analysis results are shown in FIGS. 1 to 3. The final concentration of the substrate in the reaction was 10 mM. The results of ¹H NMR spectrum analysis of the reaction products are as follows:

7,3′,4′-trihydroxyisoflavone: δ 8.37 (s, H-2), 7.97 (d, J=8.5 Hz, H-5), 7.56 (d, d, J=8.0 and 2.5 Hz, H-6′), 7.53 (d, J=2.5 Hz, H-2′), 7.42 (d, J=8.0 Hz, H-5′), 6.95 (d, d, J=8.5 and 2.5 Hz, H-6), and 6.87 (d, J=2.5 Hz, H-8).

7,5,3′,4′-tetrahydroxyisoflavone: δ 7.97 (s, H-2), δ 6.22 (d, J=2 Hz, H-6), 6.95 (d, J=2 Hz, H-2′), 6.86 (d, J=8 Hz, H-5′), 6.91 (d, d, J=2 and 8 Hz, H-6′).

### Example 2

### Examination of Changed Reactivity of Daidzein with Gram-Positive Streptomyces Avermitilis

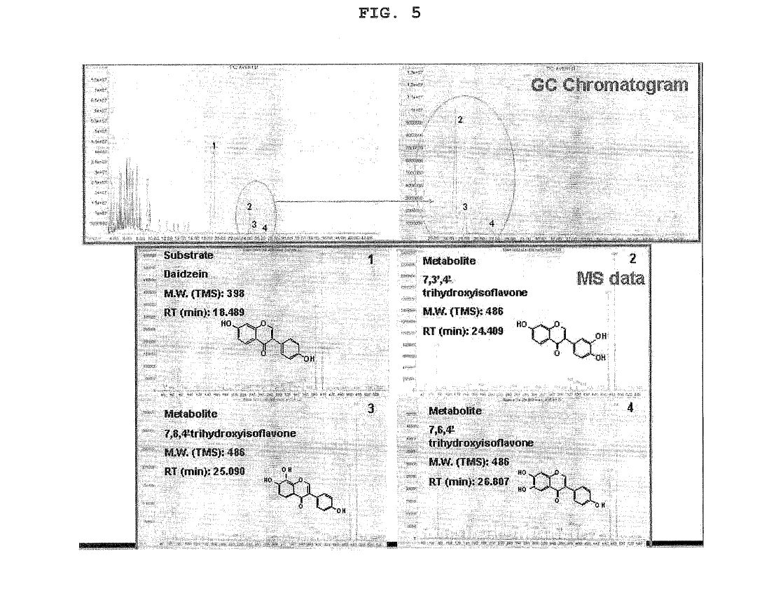

Not only hydroxylation reactivity at the 3′ position, but also hydroxylation reactivity at the 6 and 8 positions were examined using Streptomyces Avermitilis. Coils were placed for smooth oxygen supply to microorganisms, and a conical flask was used to examine the reactivity (FIG. 4(B)). An R2YE medium was used for the culture of the strain, and the reaction speed was 220 rpm. After 24 hours of the reaction, the reaction products were extracted with ethyl acetate (EA), and then analyzed using GC-MS. An authentic sample could be analyzed in the GC chromatogram through different retention times. The molecular weight of each compound could be analyzed through the MS spectrum by BSTFA derivatization, and the analysis results are shown in FIG. 5 (daidzein, room temperature, 18.5 min, MS 398; 7,3′,4′-trihydroxyisoflavone, room temperature, 24.4 min, MS 486; 7,8,4′-trihydroxyisoflavone, room temperature, 25.1 min, MS 486; 7,6,4′-trihydroxyisoflavone, room temperature, 26.8 min, MS 486).

### Example 3

### Examination of Estimated Reactivity of Daidzein with Gram-Positive Streptomyces Avermitilis

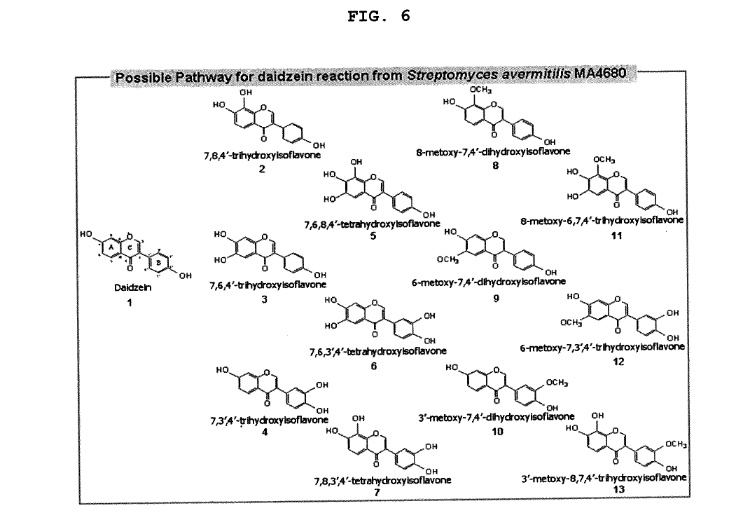

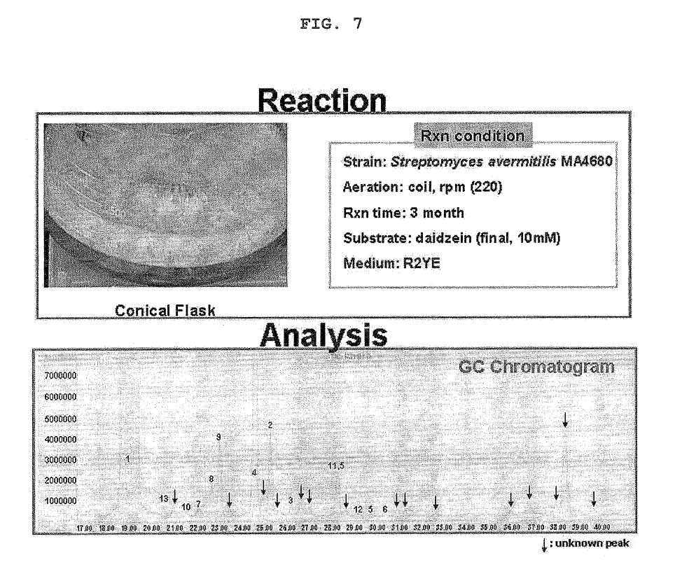

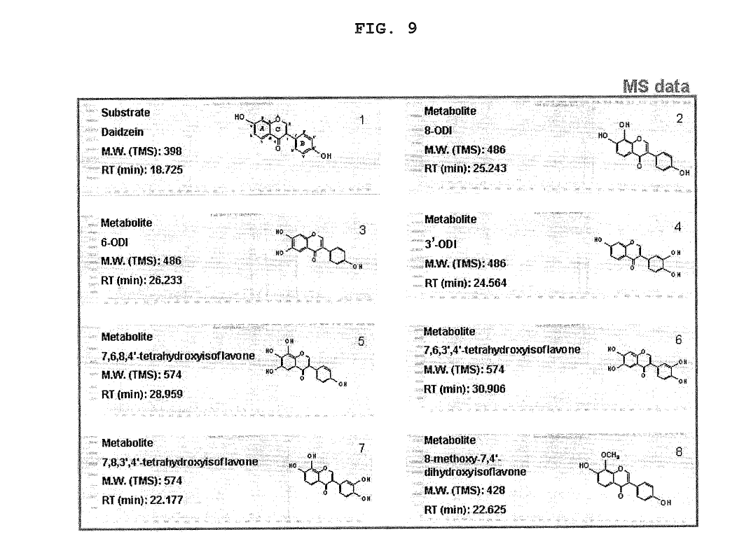

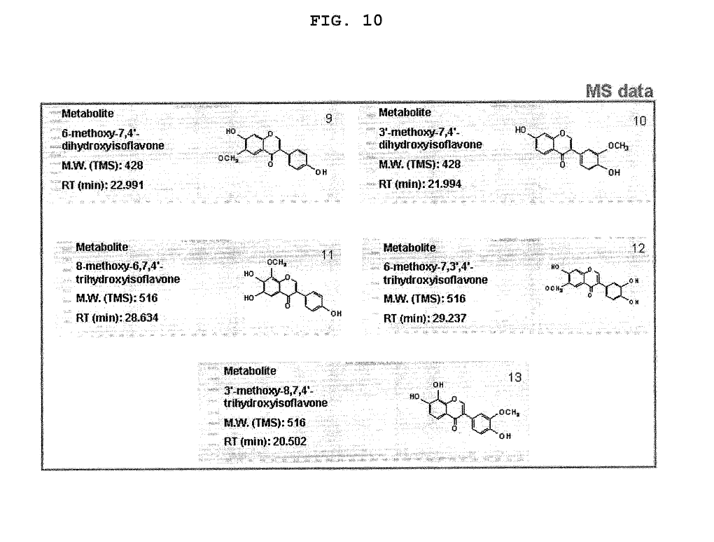

As confirmed in Example 2, Streptomyces Avermitilis had reactivity with ODI. When the reaction time was one month, various modified compounds in a monohydroxylated form, a monomethoxylated form, a dihydroxylated form, a dimethoxylated form, a trihydroxylated form and a trimethoxylated form could be analyzed by GC-MS. The results of modification degree and analysis of possible reaction products are shown in FIGS. 6 to 10. It was reported that daidzein, formononetin having methylation instead of hydroxylation at the C-4 position of B-ring of daidzein, genistein and biochanin A having methylation instead of hydroxylation at the C-4 position of B-ring of genistein have not only antioxidant effects, but also antioxidant effects upon exposure to UV light (Widyarini et al. 2001).

### Example 4

### Measurement of Melanin Production Inhibitory Effects using MeI-Ab Cells

The melanin production inhibitory effects of the ODIs obtained in Examples 1 and 2 were measured using MeI-Ab cells.

First, C57BL/6 mouse melanocyte cells (MeI-Ab cells) (Dooley, T. P. et al, Skin pharmacol, 7, pp 188-200) were cultured in a DMEM (Dulbeccos modified Eagles media) medium containing 10% fetal bovine serum, 100 nM 2-O-tetradecanoylphorbol-13-acetate and 1 nM cholera toxin in conditions of 37° C. and 5% CO₂. The cultured MeI-Ab cells were detached by 0.25% trypsin-EDTA and cultured in a 24-well plate at a concentration of 10⁵ cells/well. During the culture period, 100 ppm hydroquinone and each of the ODIs obtained in Examples 1 and 2 were added to the cells continuously during 3 days from 2 days after the start of the culture. Herein, the hydroquinone was used as a positive control group. The culture broth was collected and washed, and the cells were lysed with 1N sodium hydroxide and measured for absorbance at 400 nm. Based on the measurement results for absorbance, the melanin production inhibition of each compound was calculated according to the following Math FIG. 1, and the calculation results are shown in Table 1 below (Dooley's method).

Melanin production inhibition (%)=100−(absorbance of each test substance/absorbance of control group×100) [Math Figure 1]

TABLE 1

| | Melanin production |
|---|---|
| Test substances | inhibition (%) |
| 7,3′,4′-trihydroxyisoflavone | 38.7 |
| 7,5,3′,4′-tetrahydroxyisoflavone | 40.8 |
| 7,8,4′-trihydroxyisoflavone | 39.8 |
| 7,6,4′-trihydroxyisoflavone | 38.8 |
| Hydroquinone (positive control) | 41.1 |

As shown in Table 1 above, the ODIs obtained in Examples 1 and 2 of the present invention showed melanin production inhibitory effects similar to that of hydroquinone.

1. A method for preparing ortho-dihydroxyisoflavone, which comprises a step of biotransforming daidzein or genistein by an actinomycete microorganism.

2. The method of claim 1, wherein the biotransformation is performed by position-specific hydroxylation at the 3′ position of daidzein or genistein.

3. The method of claim 1, wherein the actinomycete microorganism is selected from the group consisting of Streptomyces avermitilis, Nocardia farcinica and Streptomyces lincolnesis.

4. The method of claim 1, wherein the biotransformation is carried out in a batch reactor into which oxygen can be supplied.

5. The method of claim 1, wherein the ortho-dihydroxyisoflavone is selected from the group consisting of 7,8,4′-trihydroxyisoflavone, 7,6,4′-trihydroxyisoflavone, 7,3′,4′-trihydroxyisoflavone and 7,5,3′,4′-tetrahydroxyisoflavone.
